(19) United States
(12) Patent Application Publication
(10) Pub. No.: US 2004/0260365 A1
(43) Pub. Date: Dec. 23, 2004

(54) Photodynamic therapy lamp

(76) Inventors: Morten Groseth, Oslo (NO); Jarle Skeidsvoll, Lonevag (NO); Torolf Wedberg, Blomsterdalen (NO); Jon Erik Braenden, Oslo (NO)

Correspondence Address: Donna M Praiss, Kenyon & Kenyon, One Broadway, New York, NY, 10004, US

(21) Appl. No.: 10/479,921

(22) PCT Filed: Jun. 7, 2002
(86) PCT No.: PCT/GB02/02704

(30) Foreign Application Priority Data
Jun. 7, 2001 (GB) 0113899.9

(51) Int. Cl. A61N 5/06

(52) U.S. Cl. 607/88; 607/90

(57) ABSTRACT

A photodynamic therapy lamp has a cover (10, 11, 12, 13) which contains a light source formed from two arrays (20) of LEDs (21). The LEDs are arranged in a honeycomb pattern and have a peak wavelength of 630-640 nm. Beneath the LEDs is a lens pack (22) containing a lens (23) for each LED. Beneath this is a diffuser (7). The lenses are arranged in a honeycomb pattern and serve to concentrate the light in a substantially parallel and narrow beam.

### FIELD OF THE INVENTION

The present invention relates to a illuminator source (also referred to as a lamp) for use in photodynamic therapy (PDT).

### BACKGROUND

Photodynamic therapy (PDT) is a developing therapy and is today used for treatment of various cancers and also for non-malignant diseases including infections, wound-healing and various dermatological diseases. The method is based on the interaction of a specific photosensitizer of oxygen and light. Clinical experience has shown that PDT has advantages over alternative therapy for treatment of several pathological conditions; including acne keratosis and various skin cancers. General background of the clinical use of PDT can be found in U.S. Pat. No. 6,225,333, U.S. Pat. No. 6,136,841, U.S. Pat. No. 6,114,321, U.S. Pat. No. 6,107,466, U.S. Pat. No. 6,036,941, U.S. Pat. No. 5,965,598 and U.S. Pat. No. 5,952,329.

Several photosensitizers are commercially available and in pre-clinical or clinical development including 5-aminolevulinic acid (5-ALA), 5-ALA derivatives and porphyrin derivatives. Other photosensitizers are suggested in the prior art, see, for example, Harat, M. et al in Neurologia i Neurochirurgia Polska 34, 973 (2000), Sharma, S. in Can. J. Ophthalmology 36, 7 (2001), Pervaiz, S. in FASEB Journal 15, 612 (2001), Korner-Stifbold, U. in Therapeutische Umschau 58, 28 (2001), Soubrane, G. et al in Brit. J. Ophthalmology 85, 483 (2001), Despettre, T. et al in J. Fr. Ophthalomologie 24, 82 (2001), Barr, H. et al in Alimentary Pharmacology & Therapeutics 15, 311 (2001), Schmidt-Erfurth, U. et al in Ophthalmologie 98, 216 (2001) and Rockson, S. G. et al in Circulation 102, 591 (2000).

One critical element in safe and efficient PDT is the light source. A clinically useful light source preferably fulfills several criteria, for instance: high intensity of the light (i.e. high radiant flux); easy to set light dose; peak wavelength of the emission spectrum within area of interest; uniform radiation light intensity within area of interest; reliable construction with low operating cost and simple construction.

There are several light sources for PDT described in prior art: U.S. Pat. No. 5,441,531 (DUSA) describes a method for PDT comprising steps involving filters and dichroic mirrors to select correct wavelengths and remove infrared radiation, U.S. Pat. No. 5,782,895 (DUSA) describes an illuminator for PDT comprising bulb holder, filters and dichroic mirror, U.S. Pat. No. 5,961,543 (Herbert Waldman) describes an apparatus for PDT irradiation with lamp reflector, filter unit and a pair of blowers, U.S. Pat. No. 5,634,711 (Kennedy) describes a hand-held portable light emitting device for PDT, U.S. Pat. No. 5,798,523 (Theratechnologies) describes a motorized device for PDT, U.S. Pat. No. 5,843,743 (Cancer Research Campaign Technology) claims a non-laser light source comprising a high intensity lamp with output intensity greater than 75 mW per square centimetre and a bandwidth in the range 0 to 30 nm, U.S. Pat. No. 5,849,027 (MBG Technologies) describes a noncoherent electromagnetic energy source being capable of generating about 300 to 400 W of broad wave length radiant energy, U.S. Pat. No. 6,007,225 (Advanced Optical Technologies) describes a directed lighting system utilizing a conical light deflector, U.S. Pat. No. 6,048,359 (Advanced Photodynamic Technologies) described apparatus and methods relating to optical systems for diagnosis of skin diseases, U.S. Pat. No. 6,096,066 (Light Sciences Limited Partnership) describes a light therapy patch, U.S. Pat. No. 6,128,525 (Zeng et al) describes an apparatus for controlling the dosimetry of PDT, WO 00/00250. (Genetronics) describes an apparatus for both electroporation of cells and light activation of the electroporated cells. WO 99/10046 (Advanced Photodynamic Technologies) describes a light emitting treatment device comprising shell and liner being made of a polymeric material. WO 98/04377 (Light Science Limited Partnership) suggest a device for applying hyperthermia to enhance the efficacy of light therapy, WO 85/00527 (M. Utzhas) describes an irradiation apparatus with a plurality of filters particularly for dermatological applications, WO 99/56827 (DUSA) describes a light source for contoured surfaces comprising a plurality of light sources, EPO 604 931 (Matushita Electric Industrial Co.) describes a medical laser apparatus, WO 99/06113 (Zeng et al) describes an apparatus for controlling the dosimetry of PDT, WO 84/00101 (The John Hopkins University) describes an apparatus for monitoring the effectiveness of PDT and prescribe a correct dosage of therapeutic photoradiation. WO 45/32441 (The Government of the United States of America) claims a light delivery device with an optical fibre, WO 00/25866 (cart) describes an apparatus for PDT using a source of non-coherent light energy with filtering and focusing means for producing radiation energy in a broad bandwidth. Other devices for photodynamic therapy are described in U.S. Pat. No. 4,576,173 (Johns Hopkins University), U.S. Pat. No. 4,592,361 (Johns Hopkins University), U.S. Pat. No. 4,973,848 (J. McCaughan), U.S. Pat. No. 5,298,742 (Dep. Health, USA), U.S. Pat. No. 5,474,528 (DUSA), U.S. Pat. No. 5,489,279 (DISA, U.S. Pat. No. 5,500,009 (Amron), U.S. Pat. No. 5,505726 (DUSA), U.S. Pat. No. 5,519,435 (Government USA), U.S. Pat. No. 5,521,392 (EFOS), U.S. Pat. No. 5,533,508 (PDT Systems), U.S. Pat. No. 5,643,334 (ESC Medical Systems Ltd.) and U.S. Pat. No. 5,814,008 (Light Science Limited Partnership).

Instead of using conventional lamps, several patents in the prior art suggest lamps for photodynamic therapy based on light emitting diodes (LEDS); WO 94/15666 (PDT Systems), FR 2492666 (Maret), WO 95/19812(Markham), U.S. Pat. No. 5,259,380 (Amcor), EP 0266038 (Kureha Kagaku Kogyo), U.S. Pat. No. 5,698,866 (PDTSystems)U.S. Pat. No. 5,420,768 (Kennedy), U.S. Pat. No. 5,549,660 (Amron) and U.S. Pat. No. 6,048,359 (Advanced Photodynamic Technologies).

There are believed to be a number of advantages in using LED technology instead of conventional lamps. For example, an array of LED's can be formed to cover a large area. In addition, their high efficiency ensures that less heat dissipation is necessary. Furthermore, LEDs have long term stability and so it is easier to design lamps which are suitable for tens of thousands of hours of operation. Other advantages include low running and maintenance costs, low driving voltage which increases safety, their mechanically robust nature, compact modular lightweight construction and ease of movement and transport.

However, despite these significant advantages, there are several disadvantages using LED technology described in the prior art for photodynamic therapy which impact on the usefulness of LED lamps in PDT.

The main disadvantage of using LED lamps in a two dimensional array is that the uniformity of the light is not good enough to obtain a safe and efficient PTD treatment. This is because the light patterns from the LED's may, for example be bat wing shaped with a wide output angle. Other disadvantages using known PTD-LED technology include: relatively high cost and complexity because a liquid-based cooling system is required, the relatively broad spectrum of light (600-700 nm) and limited amount of light output resulting in long treatment times.

### SUMMARY

According to the present invention there is provided an irradiation source for use in photodynamic therapy comprising a two-dimensional array of LEDs (light emitting diodes) and further comprising means for collimating the light emitted from the LEDs.

By collimating the light in this manner, the variation in light intensity with distance from the irradiation source is greatly reduced which means that distance between the patient and the light source does not have a critical effect on the dose received. This both simplifies the treatment and enables the effective and even treatment of non-planar surfaces. Furthermore, light intensity is increased at any significant distance from the source and the invention also enables a far more uniform irradiation pattern to be produced.

The collimation is most effectively achieved using lenses in addition to the LEDs and most preferably where each LED lamp has an associated additional lens system. In this way there may be achieved the most uniform light at any working distance from the body.

Although multi-element lenses may be used, preferably a single additional lens is provided for each LED. The preferred lens for use in the present invention is a lens able to direct the light as to secure uniform light intensity over area of interest. Typical lenses are lenses made of synthetic materials or glass. The most preferred lens type is an axicon collimating lightguide. It is most preferred that such a lens is designed to reduce scattering effects which would otherwise cause light to be lost outside of the otherwise near collimated beam.

Although the arrangement so far described provides significant benefits over the prior art, to further ensure an even broader field of light of homogeneous character, the lens system is preferably made up of hexagonal lens units which may be closely packed together in a hexagonal pattern, preferably on the diode matrix. Thus, the individual lenses are preferably hexagonal, or substantially hexagonal in plan. From a further aspect the invention, this provides a PDT lamp comprising an array of generally hexagonal lenses arranged in a honeycomb pattern. Each lens preferably abuts the adjacent lenses.

The change in light intensity over area of interest should be less than ±15%, preferably less than +/10%, most preferably less than ±7%.

Although lower outputs may be used if desired, the source according to the present invention preferably gives at least 20 mW/cm². It is also preferred that output is no more than 100 mW/cm² at a nominal distance of 5 cm based on a Full Width Half Maximum (FWHM) of about 18 nm. Preferably the output is more than 40 mW/cm² at 5 cm distance to avoid long treatment times.

The number of LEDs may be varied depending on irradiation area, although a practical number of LEDs lies between 1 and 3000. The more preferable number would be between 4 and 512 and the most preferable number would be between 8 and 256 LED's.

The irradiation area may be varied depending upon the lens arrangement and the number of LEDs, but this is preferably between 1 m² and 3000 cm².

A lamp for irradiation of 40 mm×50 mm may for example have 16 diodes. A lamp for irradiation of 90 mm×190 mm may for example have 128 diodes. The distance between the diodes is preferably in the range of from 2 mm to 20 mm; depending upon light intensity.

To be useful in PDT, the peak wavelength of the light is preferably in the range 620-645 nm, more preferably 625-640 nm and most preferably 630-640 nm, for example for use with Photoporphyrin IX. However, the lamp can have different wavelengths—with different LEDs to cover the peak areas of other photosensitizers like Photofrin, Phorphycenes, Sn-Etiopurin, m-THPC, NpE6, Zn-Phtalocyanine and Benzoporphyrin.

Although an LED based lamp generates less heat itself than other types of light source, the lamp may optionally be equipped with patient fan for cooling of the patients target area. Preferably this is combined with the cooling system for the lamp itself. Thus, for example, the lamp may be provided with a cooling fan which directs air both to cool the LEDs (either directly or indirectly) and out of the lamp in the same general direction as the emitted light such that the irradiated part of the patient may be cooled. For example, air drawn into the lamp by the fan may be divided into two streams, one for each purpose.

The diodes are preferably associated with a heat sink to dissipate heat and this may in turn be cooled by an airstream provided by a fan. This may be continuous or controlled by a simple thermostatic switch, but preferably this is microprocessor controlled, e.g. based upon input from a temperature sensor. If necessary, the temperature of the LEDs may be controlled in order to vary peak output frequency. Such control may be provided by means of a NTC resistor, e.g. providing an input to the microprocessor. A typical frequency variation is 0.2 nm/K.

This concept is itself believed to be inventive and so viewed from another aspect there is provided a light source for use in PDT wherein the light source comprises an array of LEDs and the output frequency of the LEDs is varied by controlling their temperature.

Preferably the lamp is microprocessor controlled, such that, additionally or alternatively, there may be provided a dose timer and/or a timer for determining the life of the lamp (based upon total usage time). There may also be provided automatic distance measurement equipment such that the irradiation dose may be adjusted (automatically or manually) to correct for the remaining variation of intensity with distance from the source.

Also, there may be provided means for modulation of the light source, again preferably under microprocessor control, such that the amplitude or frequency of the light may be varied over time, e.g. in accordance with a program stored in computer memory. Such modulation may provide for more effective treatment in certain situations. For example, it is thought that a pulse train of light followed by a brief pause will allow the cells to pick up more oxygen. Preferably the modulation is user-programmable. The provision of a modulatable lamp (preferably as just described) forms another aspect of the invention. Thus, viewed from another aspect the invention provides a lamp for use in PDT having a plurality of LED light sources which are modulatable in use.

A further preferred feature is the provision of segmentation means for reduction of illuminated area. Thus, for example, either e.g. 8 groups LEDs may be selectively de-activated, or masks may be provided within the lamp to prevent light from selected LEDs from reaching the patient.

Although the light provided by means of the invention, and particularly in its preferred forms will be sufficiently uniform for any PDT application, uniformity may be still further improved by providing for the mechanical oscillation of the LEDs such that each collimated beam is moved over the target surface. It will be appreciated that only a small degree of movement maybe employed, for example to enable the optical axis of one beam to travel halfway towards a point defined on the target by the previous position (e.g., before movement) of the optical axis of an adjacent beam. Again, this concept forms another aspect of the invention and so viewed from another aspect there is provided a lamp for use in PDT comprising an array of light sources which are arranged to oscillate. The invention also extends to a method of providing PDT and so viewed from a still further aspect the invention provides a method of PDT comprising the use of a lamp or light source according to any other aspect of the invention. Preferably the method comprises the use of a lamp or source according to any of the preferred forms of the invention.

### BRIEF DESCRIPTION OF THE DRAWINGS

Certain embodiments of the invention will now be described, by way of example only, and with reference to the accompanying drawings:

FIG. 1 is a perspective view of a first embodiment of the invention showing its mounting arm, in accordance with one embodiment of the present invention;

FIG. 2 is a perspective view from below of the embodiment of FIG. 1;

FIG. 3 is a perspective view from above of the embodiment of FIG. 1;

FIG. 4 is an exploded view (corresponding to FIG. 2) of the embodiment of FIG. 1;

FIG. 5 is an exploded view from beneath and one side of the embodiment of FIG. 1;

FIG. 6 is an exploded view from beneath and the other side of the embodiment of FIG. 1;

FIG. 7 is a perspective view from above of another embodiment of the invention showing its mounting arm;

FIG. 8 is a perspective view from below of the embodiment of FIG. 7;

FIG. 9 is a perspective view from above of the embodiment of FIG. 7;

FIG. 10 is an exploded view from above of the embodiment of FIG. 7;

FIG. 11 is an exploded view from below of the embodiment of FIG. 7;

FIG. 12 is a schematic ray diagram illustrating the optics, in accordance with one embodiment of the present invention;

FIG. 13 is a schematic view illustrating the arrangement of LEDs, in accordance with one embodiment of the present invention;

FIG. 14 is a perspective view of a lens, in accordance with one embodiment of the present invention;

FIGS. 15a and 15b illustrate the effect of the lenses, in accordance with one embodiment of the present invention; and

FIG. 16 illustrates the effect of varying LED junction temperature on peak wavelength.

### DETAILED DESCRIPTION

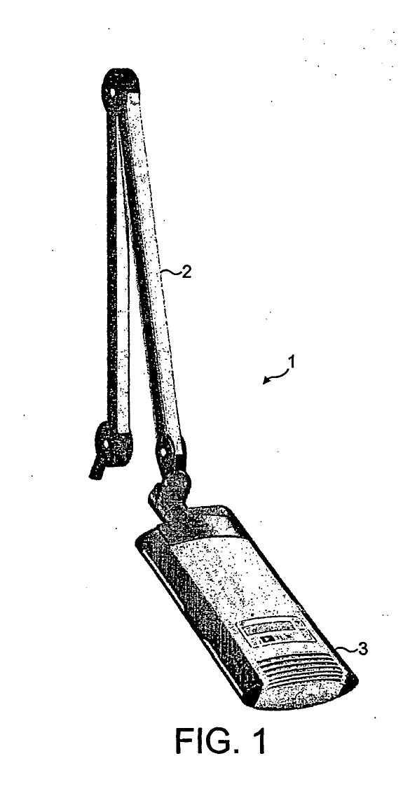

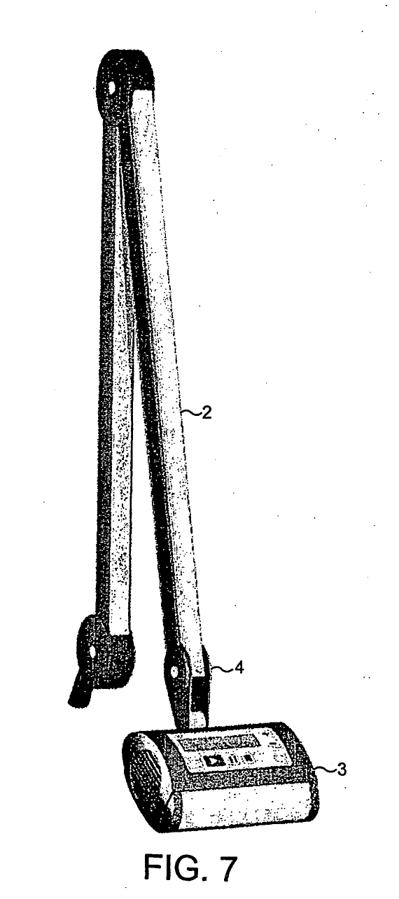

With reference first to FIG. 1, a phototherapeutic lamp 1 consists of a supporting counterbalanced arm 2 with clamp (not shown), an external power supply (not shown), and a lamp head 3. This Figure shows the first embodiment of the invention, but the second embodiment is also provided with a similar arm (see FIG. 7). The arm enables the lamp to be secured to a table-like surface, for example in a physician's consulting room. The arm is essentially conventional and allows the lamp head to be moved into position over a part of a patient's body that is to be treated.

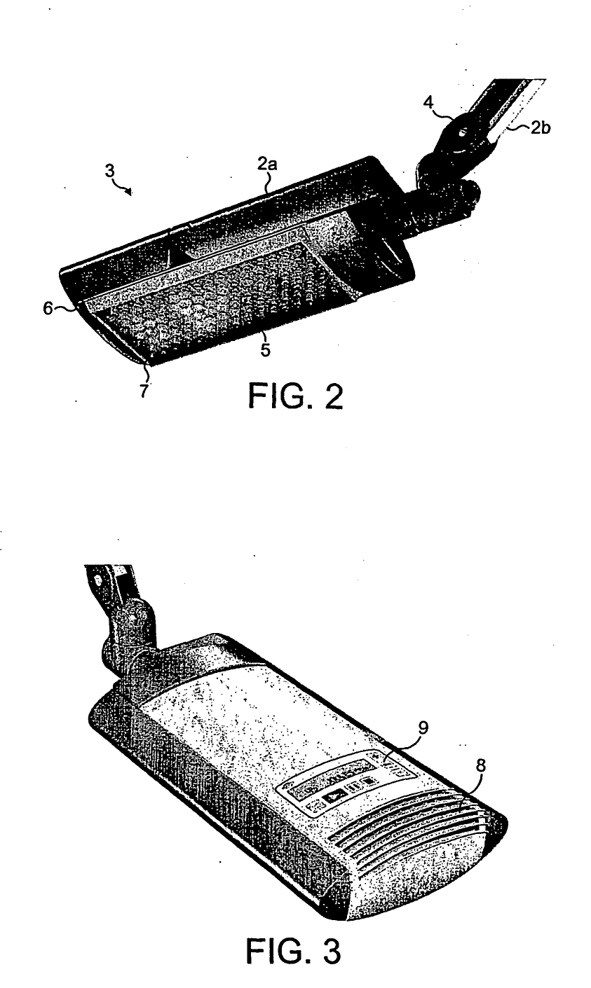

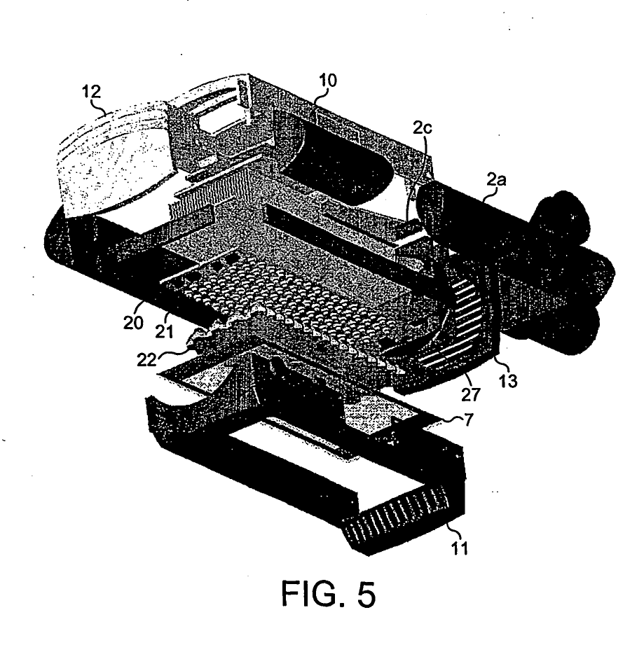

Turning now to FIG. 2, the lamp head 3 of the first embodiment can be seen to be pivotally mounted to a side arm 2a which is shaped to conform generally to the outer shape of the lamp head. (This may be seen more clearly in FIG. 5 where it may be seen that side arm 2a engages with pivot pin 2c.) The side arm is itself connected to main arm 2b via a swivel joint 4. Swivel joint 4 allows for movement about two perpendicular axes and the pivotal mounting of the side arm to the lamp head provides for additional movement.

Housing 6 has an opening in its lower surface where the light source 5 is visible through thin diffuser 7. From FIG. 3 it may be seen that the upper part of the housing 6 is provided with an air outlet 8 in the form of ventilation slots formed in the housing itself. There is also a control panel and display unit 9.

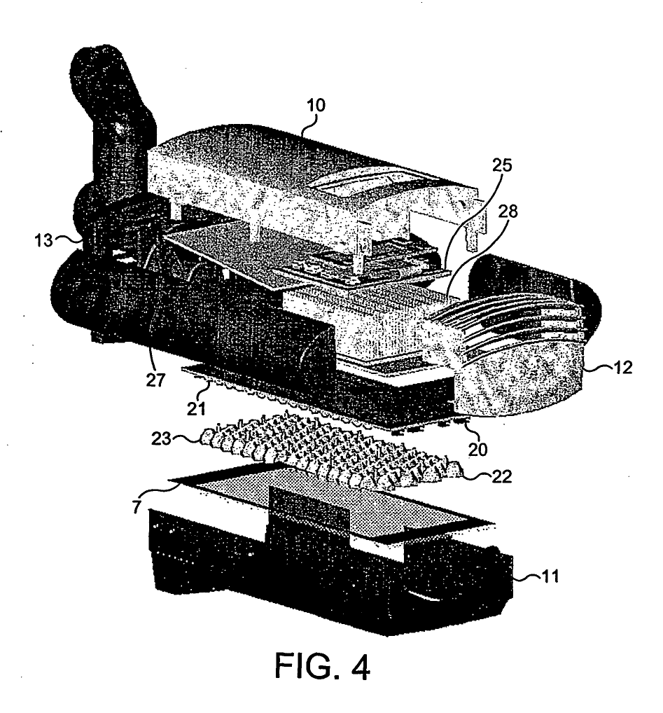

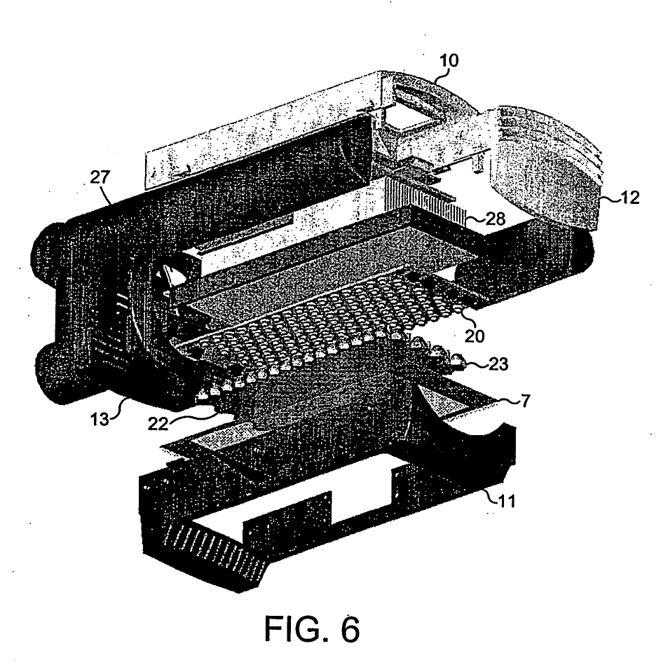

With reference now to FIGS. 4 to 6, it may be seen that the housing 6 is formed from several molded plastic components: the upper cover 10, the lower cover 1 1, and end covers 12 and 13. Both end covers are provided with ventilation slots to allow for a flow of air through the lamp in use, those on end cover 13 being an air intake and those on end cover 12 being the outlet.

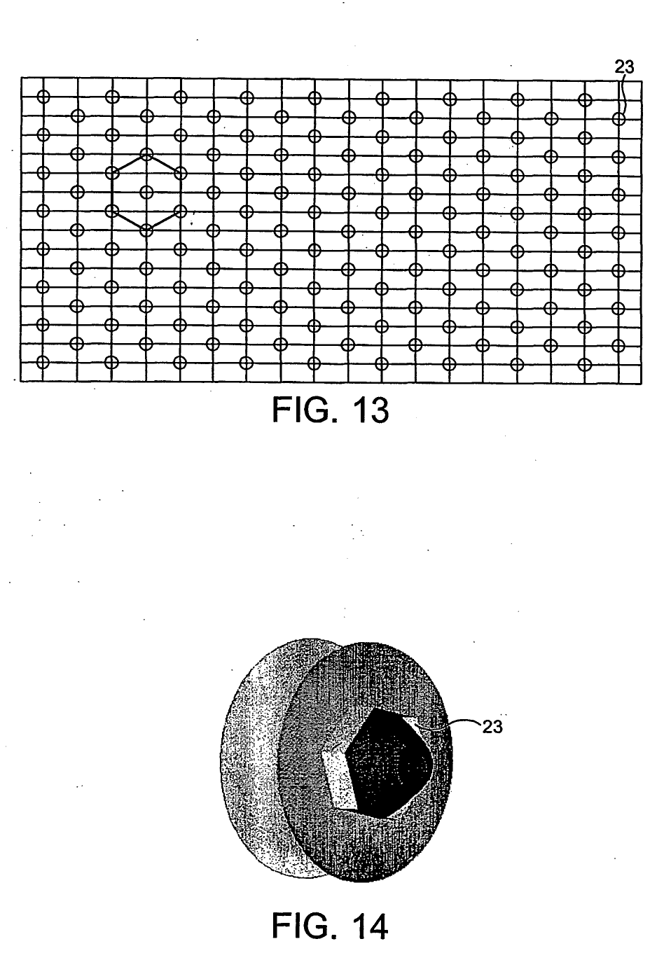

Within the housing there is a light source made up of several LED's, a control unit, a cooling system and a lens system provided within a housing. These components will be discussed in more detail below. The light source is formed from an a two arrays 20 of modules each containing 64 LEDs 21. The LEDs are arranged in a honeycomb pattern (i.e. a hexagonal array) as illustrated in FIG. 13. The LEDs each have a peak wavelength in the range 630-640 nm and an output of 60 W/cm2 at 5 cm.

Beneath the LED arrays 20 is a lens pack 22 containing a lens 23 for each LED. Beneath this in turn is thin diffuser 7 which is located in a recess in an opening in the lower cover 11.

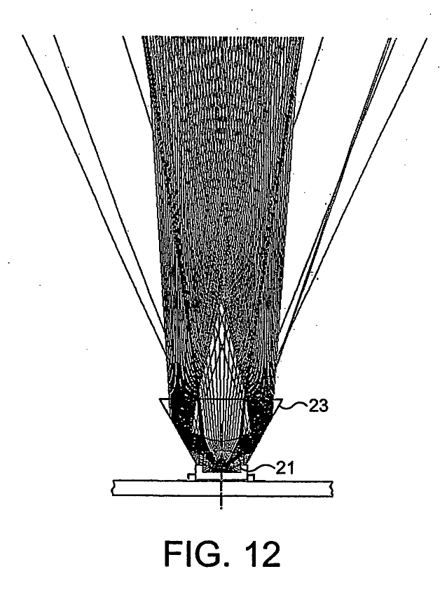

FIG. 14 illustrates one of the lenses 23 and FIG. 12 is a ray diagram showing its operation. The LED 21 is at the bottom of the Figure with the lens 23 above it. The diffuser 7 has been omitted in the interests of clarity. As may be seen from the ray diagram, substantially all of the light from the LED 21 is concentrated in a substantially parallel and narrow beam centred on the optical axis of the lens and LED.

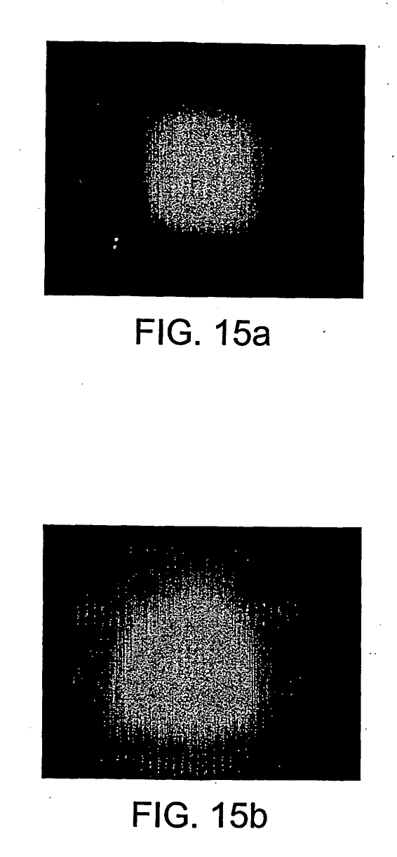

As will be discussed below, the effect of the lenses is illustrated in FIGS. 15a and 15b.

The current to the LED modules is supplied by the power supply which is conventional and will therefore not be described further via a microprocessor-based control unit 25. As well as controlling the supply of current to the LEDs 21, the control unit also controls electric cooling fan 27 and various other features such as a lamp-life monitor, dose timer, etc.

In order to maintain the desired output radiation frequency, it is important that the LED's 21 do not get too warm but can be controlled at a relatively stable temperature. Hence the fan is part of an air cooling system which further comprises a heat sink 28 mounted to the back of the LED panels. The fan forces the air to move in through air intake in cover 13, over the LED arrays 20 and out via the outlet in cover 12 through the cooling ribs. The operating temperature is sensed via a sensor (not shown) and a feedback system is provided such that the microprocessor controls this temperature.

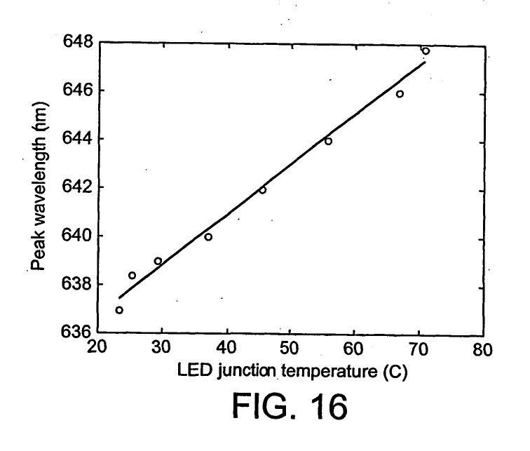

If necessary, the temperature of the LEDs can be varied in order to adjust the output peak wavelength of the LEDs. There is an approximately linear relationship between LED junction temperature and wavelength. FIG. 16 illustrates the result of an experiment to demonstrate this. In this experiment, the LED-spectra at different LED junction temperatures were recorded and the peak wavelength was plotted versus LED junction temperature. This is shown in FIG. 16 where it can be seen that the peak wavelength is proportional to the junction temperature. A best linear fit to the data points gives a proportionality of 0.208 nm per degree C. Thus, the junction temperature may be controlled in the LED lamp ensure an overlap between the absorption spectrum of the photosensitizer (e.g. protoporphyrin IX) and the LED emission spectrum.

The airstream is in fact split into two paths at the intake. One path is directed to the heat sink 28 and the other path is arranged to blow air over the patient's skin. This provides a cooling effect which reduces the pain introduced by the reaction of the chemical drug.

In use, the lamp is secured to a surface via the arm 2a, 2b and the clamp (not illustrated). The lamp is then positioned over the area of the patient's skin that is to be irradiated.

The controls for the lamp are found in control panel/display unit 9.

The system is switched on and off by pressing the ON/OFF button. When turning the system on, the button is pressed and held it until the text “CURELIGHT V x. x, Ser. no: 0100XXXX” appears in the display window. The button is then released. After a few seconds, the message “REMAINING LAMP LIFE: XXhXX” is displayed. This shows the remaining FULL LIGHT operative time, as calculated by the microprocessor, displayed in hours and minutes. When the timer shows Oh00, no further use is possible. A dose timer is also provided which indicates how much longer the lamp will operate during a particular treatment.

The system is switched off by pressing the ON/OFF button once more. Pressing the button gives a beep, and the system is switched off.

In order to correctly position the lamp over the area to be treated the operator presses the GUIDE LIGHT button to switch on the lamp with low power. The lamp may then be moved such that the correct area of skin is under illumination. The timers will not be affected in LOW LIGHT mode, even though the current value of the dose timer will be shown. Normally, this timer will be 0:00, unless an ongoing FULL LIGHT treatment has been halted. By pressing the GUIDE LIGHT button once more, the light is switched off.

If the lamp was in FULL LIGHT mode prior to pressing the GUIDE LIGHT button, the lamp switches to GUIDE LIGHT and the timers will stop.

In addition a PAUSE button is provided which can be used to temporarily stop the treatment. Pressing this button again will continue the treatment from where it left.

There is also a MODE BUTTON which is used to select a SET DOSE function in order to adjust the light dose if necessary. The buttons are used together with the SET DOSE function to adjust the dose value. The ± buttons adjust the dose in steps of 1 J/cm², and the corresponding dose time will be calculated and displayed simultaneously as minutes and seconds. By holding the buttons down a rapid up or rapid down adjustment will occur. It is believed that a light dose of 37 J/cm² is most effective. The Mode button can also be used to activate other functions like decreasing segments of the illuminated area (less treatment area).

After the lamp has been correctly arranged, the operator presses the START button to switch the lamp to therapeutic intensity. The dose timer and the lamp timer count down when the lamp is in FULL LIGHT mode. Only the dose timer is displayed.

When the dose timer comes to 0:00, the light is automatically switched off and the flashing message “END OF DOSE” is displayed. A pulsing sound is emitted until the RESET button (see below) is pressed.

The STOP/RESET button can be used to abort an ongoing operation or to clear an “END OF DOSE” or error message.

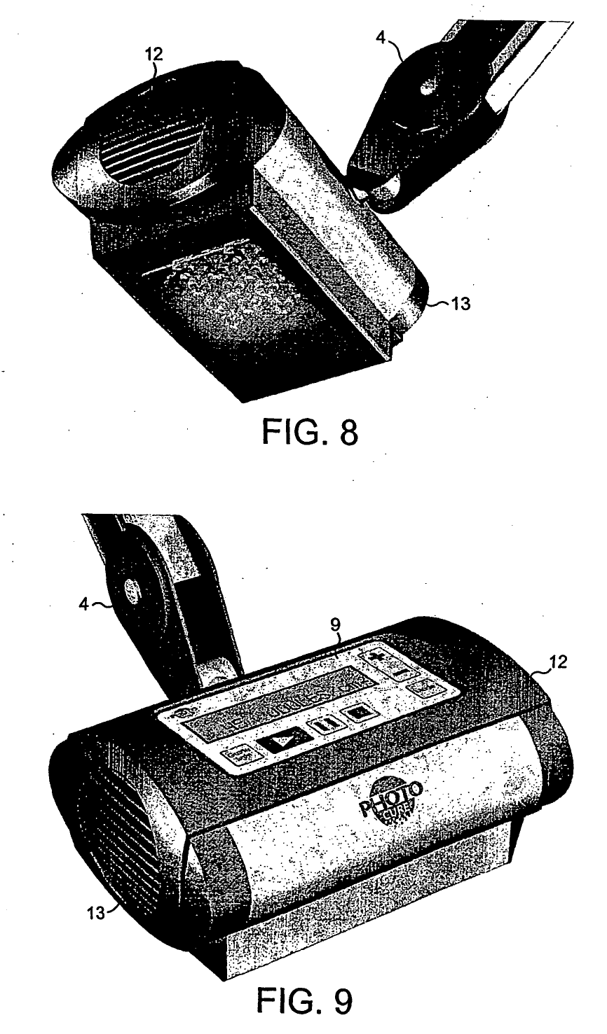

The second embodiment of the invention is in most operational respects similar to the first, although, as may be seen from FIGS. 7 to 11 it has a rather different appearance and structure. In particular, the housing is effectively rotated by 90 degrees such that the arm 2 is connected via swivel joint 4 directly to the side of the housing, without the use of a side arm. Additionally, the air intake and outlet are provided in the end covers 12, 13 which are here found at opposite sides of the joint 4.

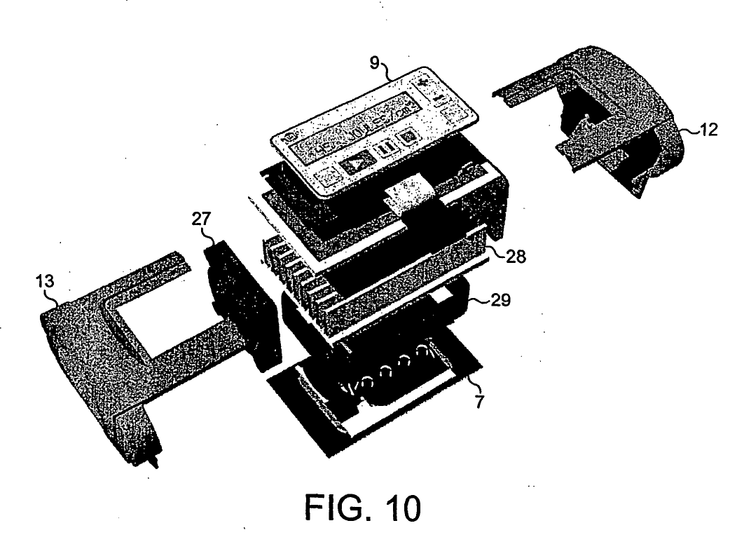

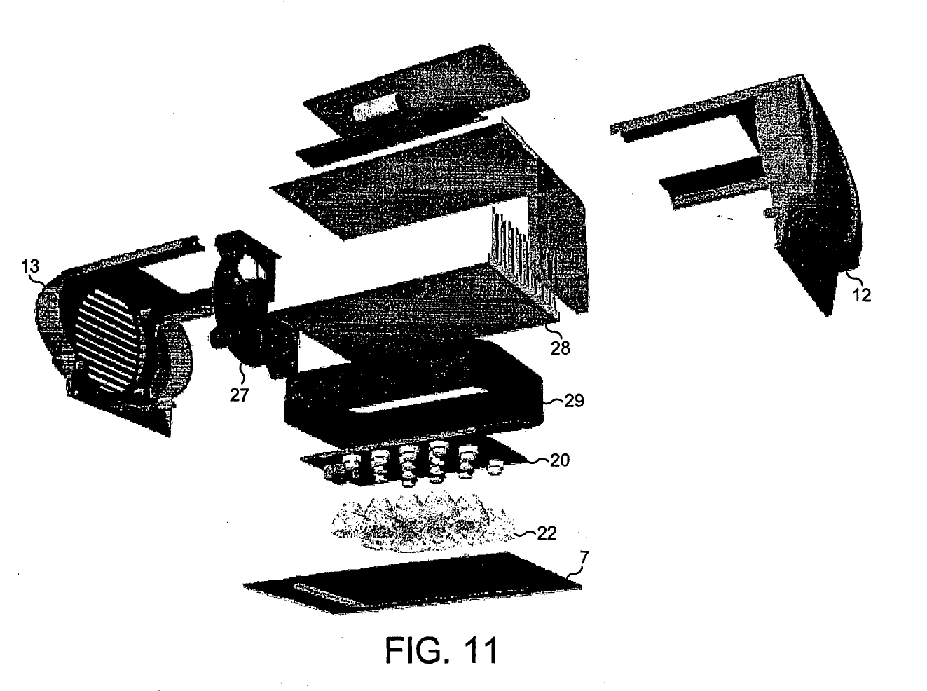

As may be seen from FIGS. 10 and 11, the lamp head 3 has a housing formed from the two end covers 12, 13 and front and back covers (not shown in these Figures for reasons of clarity).

FIG. 11 best illustrates the light-source arrangement which, like the previous embodiment comprises a thin diffuser 7, a lens array 22, LED array 20 and heat sink 28. It will be noted, however, that the number of LEDs and lenses is much reduced and so it will be appreciated that this lamp is intended for use on smaller areas of skin. Forming an additional part of the cover is light surround 29.

Towards the left-most side of the Figure, fan 27 draws air in though the intake and directs it over the a fins of the heat sink 28, as previously discussed.

Above the heat sink the control system and display are provided—these may more clearly be seen from FIG. 10.

The lamp of the second embodiment it operated in an identical manner to that discussed above in relation to the first embodiment.

Finally, an example of one of the lenses used in both embodiments is illustrated in FIG. 14. It will be noted that the lens has a hexagonal outer form in order to enable it to be packed in the hexagon (honeycomb) arrangement illustrated in FIG. 13. The lens is an axicon collimating lightguide and shaped such that it provides a substantial collimated beam as shown in FIG. 12.

FIGS. 15a and 15b illustrate the result of an experiment to demonstrate the effect of lens arrays 22. Two LED arrays with (FIG. 15a) and without (FIG. 15b) lenses were placed under frosted glass and photographed at the same distance between the frosted glass and camera. It can be seen from FIG. 15a that the lenses concentrate the light into a defined field, whereas in FIG. 15b the light is much more dispersed.

As previously discussed, because the beam is effectively collimated the distance between the lamp and the patient is not critical to the dose (light energy) delivered. Not only does this mean that the lamp does not have to be located a precise distance from the patients skin, it also means that non-planar surfaces may be effectively treated without significant variation in dose between raised and lower areas.

Several embodiments of the present invention are specifically illustrated and/or described herein. However, it will be appreciated that modifications and variations of the present invention are covered by the above teachings without departing from the spirit and intended scope of the present invention.

What is claimed is:

1-14. (Cancelled).

15. An irradiation source for use in photodynamic therapy comprising:
a two-dimensional array of light emitting diodes; and
means for collimating the light emitted from the light emitting diodes.

16. The irradiation source as claimed in claim 15, wherein each light emitting diode has an associated additional lens system.

17. The irradiation source as claimed in claim 16, wherein a single additional lens is provided for each light emitting diode.

18. The irradiation source as claimed in claim 17, wherein the lenses are one of hexagonal and substantially hexagonal in plan view.

19. The irradiation source as claimed in claim 18, wherein the lens system includes hexagonal lens units which are arranged together in a hexagonal lens units which are arranged in a hexagonal pattern.

20. The irradiation source as claimed in claim 15, further comprising:
a microprocessor; and
at least one of a dose timer and a timer for determining a life of the light emitting diode, wherein the at least one of a dose timer and a timer for determining a life of the light emitting diode is controlled by the microprocessor.

21. The irradiation source as claimed in claim 15, further comprising:
a fan for cooling a patient's target area.

22. The irradiation source as claimed in claim 21, wherein the fan operates to cool the light emitting diodes.

23. An irradiation source as claimed in claim 22, wherein the irradiation source includes a cooling fan which directs air both to cool the light emitting diodes and out of the lamp in the same general direction as the emitted light so as to cool an irradiated part of the patient.

24. The irradiation source as claimed in claim 15, wherein an airstream is provided to control the temperature of the diodes, the airstream being microprocessor controlled.

25. The irradiation source as claimed in claim 15, wherein the output frequency of the light emitting diodes is varied by controlling their temperature.

26. The irradiation source as claimed in claim 15, wherein the irradiation source is modulatable.

27. The irradiation source as claimed in claim 26, wherein the amplitude or frequency of light is modulatable under microprocessor control.

28. A method of photodynamic therapy comprising the use of an irradiation source according to claim 15.
